(19) United States
(12) Patent Application Publication
(10) Pub. No.: US 2026/0215553 A1
(43) Pub. Date: Jul. 30, 2026

(54) ACTIVE AND PASSIVE COMPANION RINGS FOR WEARABLE DEVICES

(71) Applicant: Oura Health Oy, Oulu (FI)

(72) Inventors: Jukka-Tapani Mäkinen, Oulu (FI); Lillian Moore Hackett, San Francisco, CA (US); Nuutti Mikael Vienola, Oulu (FI)

(73) Assignee: Oura Health Oy, Oulu (FI)

(21) Appl. No.: 19/040,031

(22) Filed: Jan. 29, 2025

(51) Int. Cl. A44C 9/00; A61B 5/00

(52) U.S. Cl. CPC A44C 9/0053; A61B 5/0059; A61B 5/6826

(57) ABSTRACT

Systems and methods are disclosed for active and passive companion rings for wearable devices. In one embodiment, a set of wearable ring devices includes a first ring having a first housing, where the housing comprises a first surface reflectance value between about 5% and about 100%, and a second ring disposed adjacent to the first ring. The second ring can include a second housing, one or more optoelectronic light source components, and an optoelectronic component configured to detect light output from at least one of the one or more optoelectronic light source components.

### BACKGROUND

Users of wearable devices may utilize such devices for various purposes, such as tracking movement, biometrics, activity levels, and so forth. Wearable devices in different form factors, such as rings, watches, and the like may be used. Different form factors have different technical challenges related to device performance. For example, wearable devices in a watch form factor may not consistently be in contact with a user's skin, whereas wearable devices in a ring form factor may be uncomfortable if too bulky. Moreover, for wearable devices in a ring form factor, users may wear additional jewelry in conjunction with the wearable device in the ring form factor. Such additional jewelry may impact performance of the wearable devices in ring form factor. Accordingly, active and/or passive companion rings for wearable devices may be desired.

### BRIEF DESCRIPTION OF THE DRAWINGS

FIG. 1 is a hybrid schematic illustration of an example use case for active and passive companion rings for wearable devices in accordance with one or more embodiments of the disclosure.

FIGS. 2A-2B are schematic illustrations of example use cases for active and passive companion rings for wearable devices in various configurations in accordance with one or more embodiments of the disclosure.

FIG. 3 is a schematic illustration of example wearable device sensor operation in accordance with one or more embodiments of the disclosure. FIG. 4 depicts graphs representing example data related to the simulated impact of companion rings to sensor data in accordance with one or more embodiments of the disclosure. FIG. 5 is a schematic illustration of an example process flow for automated calibration curve selection based on a detected companion ring in accordance with one or more example embodiments of the disclosure.

FIG. 6 depicts schematic illustrations of a wearable device and an active companion ring in both a coupled and an uncoupled configuration in accordance with one or more embodiments of the disclosure.

FIGS. 7A-7B schematically illustrate example computer system architecture in accordance with one or more embodiments of the disclosure. The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION

### OVERVIEW

Users may utilize smart or active wearable devices in a ring form factor to track various user data, such as the user's activity level, sleep metrics, stress levels, physiological metrics, and/or other data. Such ring devices may be worn on a user's finger. A ring form factor may be less intrusive or bothersome to a user relative to larger form factors, such as watches, bands, etc. Moreover, wearable devices in a ring form factor may be aesthetically pleasing to users. Some users may wear wearable ring devices in conjunction with additional jewelry, such as additional rings. Such additional rings may be passive, or traditional, jewelry in some instances. For example, a user may wear a decorative jewelry ring adjacent to a wearable ring device on the user's finger. Some users may wear more than one ring on the user's finger in various configurations, such as adjacent to both sides of a wearable device ring. Such rings may be for fashion or style purposes. In some instances, users may wear more than one wearable device ring adjacent to one another, such as a first ring having sensors and a second ring having a battery and/or additional hardware. Additional configurations are possible. In instances where a ring is worn adjacent to a wearable ring device, or a "smart" ring, the adjacent ring may impact sensor performance of one or more sensors of the wearable ring device. For example, a light-colored metal ring worn adjacent to a wearable ring device may impact sensor performance for sensors that use light-based measurement techniques, such as one or more light emitting diodes and corresponding photodetectors. This is due in part to reflective properties of the material and/or color of the adjacent ring. When light is emitted and used for certain measurements by a wearable ring device, some light may escape through an interface between the wearable ring device and the user's finger. Such light loss may be accounted for using one or more calibration curves, where impact on measured value can be mitigated. However, when an adjacent ring reflects escaped light back into the wearable ring device (e.g., through the user's finger, etc.), such reflected light may interfere with accurate sensor measurements.

Embodiments of the disclosure include active and passive companion rings for wearable devices, such as wearable ring devices, that mitigate the impact of reflected light on sensor performance, and in some instances, enhance sensor performance. For example, using certain types of materials and/or colors to form companion or adjacent rings, sensor performance can be improved by controlling reflective properties. Some embodiments include wearable ring devices that can dynamically detect reflected light and/or reflective properties of companion rings and select corresponding calibration curves to offset impact of reflected light, thereby allowing users to change companion rings for aesthetic or other purposes as the user desires. Some embodiments include active companion rings that can enhance wearable ring device performance by providing additional and/or different functionality, power reserve, and/or other technical advantages. Embodiments may therefore improve the reliability, manufacturability, and performance of wearable devices.

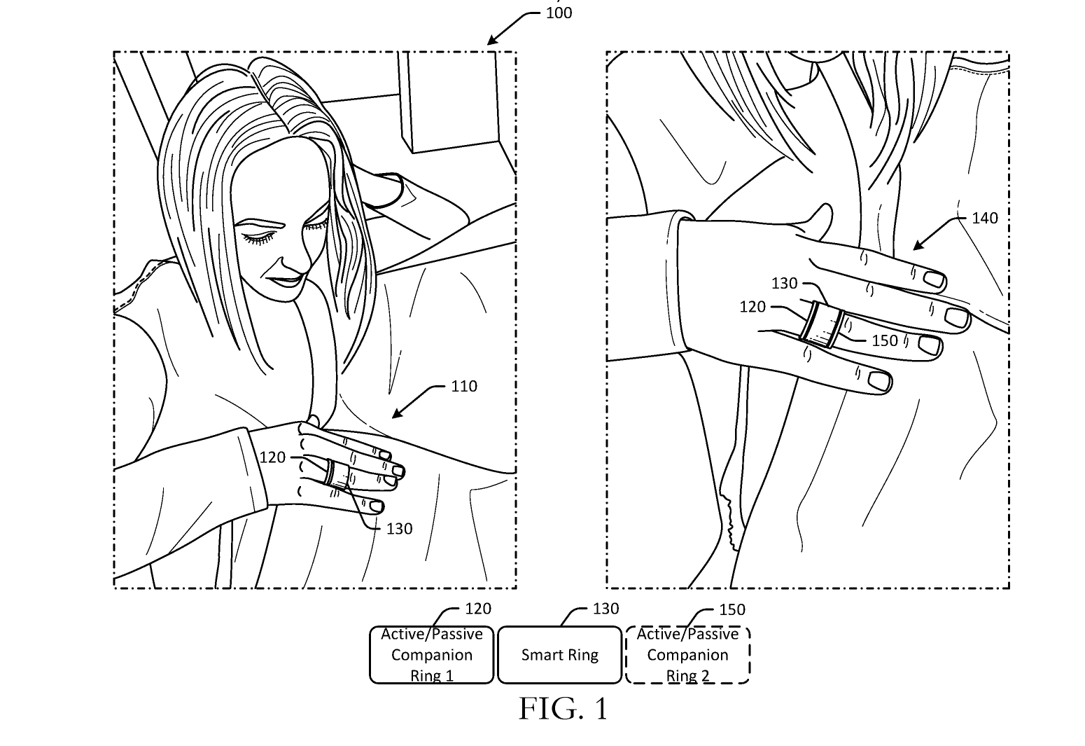

Referring to FIG. 1, an example use case 100 for active and passive companion rings for wearable devices in accordance with one or more embodiments of the disclosure. Although discussed in the context of wearable ring devices, other embodiments may be directed to any suitable use case where wearable devices are used, such as wristwatches, bands, and so forth. In FIG. 1, at a first instance 110, a user may be wearing more than one ring on a single finger. For example, the user may wear a smart ring, or a wearable ring device, 130 on a finger. The user may wear a first companion ring 120 adjacent to the wearable ring device 130 on the same finger. The first companion ring 120 may be an active companion ring, such as a companion ring that includes one or more electronic components, or a passive companion ring, such as a decorative jewelry piece. At a second instance 140, the user may be wearing more than two rings on the same finger. For example, the user may wear the first companion ring 120, the wearable ring device 130, and may also wear a second companion ring 150 on the same finger. The second companion ring 150 may be an active companion ring, such as a companion ring that includes one or more electronic components, or a passive companion ring, such as a decorative jewelry piece. The second companion ring 150 may be disposed on a first side of the wearable ring device 130, and the first companion ring 120 may be disposed on a second side of the wearable ring device 130. The companion rings may be arranged in a stacked configuration, as depicted in the examples of FIG. 1.

One or both the companion rings may impact performance of sensor(s) or measurements collected or otherwise determined by the wearable ring device 130. In particular, sensor measurements related to photoplethysmography (PPG), which can be used to determine a heart rate of the user, can be impacted by companion rings. This is due to interference with PPG sensor optical paths through a skin of the user. Different colors and/or materials of companion rings may distort PPG signals as light reflects from the outer surface of the companion ring back to one or more sensors of the wearable ring device 130. Other sensor measurements, such as heart rate variability, blood oxygen saturation, etc. may be improved by one or more embodiments as described herein.

The closer a companion ring is to an optical path used by the wearable ring device 130, the larger the potential impact of the companion ring on a corresponding sensor measurement. In particular, red and infrared signals travel relatively longer distances inside the tissue of the user's finger, and optical paths are not limited to the area of the finger covered by a width of the wearable ring device 130, allowing for greater potential impact to sensor measurements. Moreover, exposed optoelectronic components, such as light emitting diodes and/or photodetectors, are more sensitive to stray light. With reduced widths of wearable ring devices, the risk of exposed optoelectronic components increases.

Embodiments address these and other issues by, in one example, providing passive companion rings that enhance PPG sensor measurements via controlled reflections. Certain embodiments include companion rings that have a flat surface reflectance spectra, so as to support blood oxygen saturation measurements. Other embodiments may have a high surface reflectance spectra, so as to improve signal quantity and/or quality. Some embodiments facilitate the use of inner housing (e.g., an inside surface of the wearable ring device 130 that contacts the finger of the user, etc.) of a same color as an outer housing, in contrast to typical wearable ring devices that have metal inner housings to assist with light reflection.

Example embodiments of the disclosure provide a number of technical features or technical effects. For example, in accordance with example embodiments of the disclosure, certain embodiments may improve processing speed, sensor measurement, and/or device performance. The above examples of technical features and/or technical effects of example embodiments of the disclosure are merely illustrative and not exhaustive.

One or more illustrative embodiments of the disclosure have been described above. The above-described embodiments are merely illustrative of the scope of this disclosure and are not intended to be limiting in any way. Accordingly, variations, modifications, and equivalents of the embodiments disclosed herein are also within the scope of this disclosure. The above-described embodiments and additional and/or alternative embodiments of the disclosure will be described in detail hereinafter through reference to the accompanying drawings.

### ILLUSTRATIVE EMBODIMENTS AND USE CASES

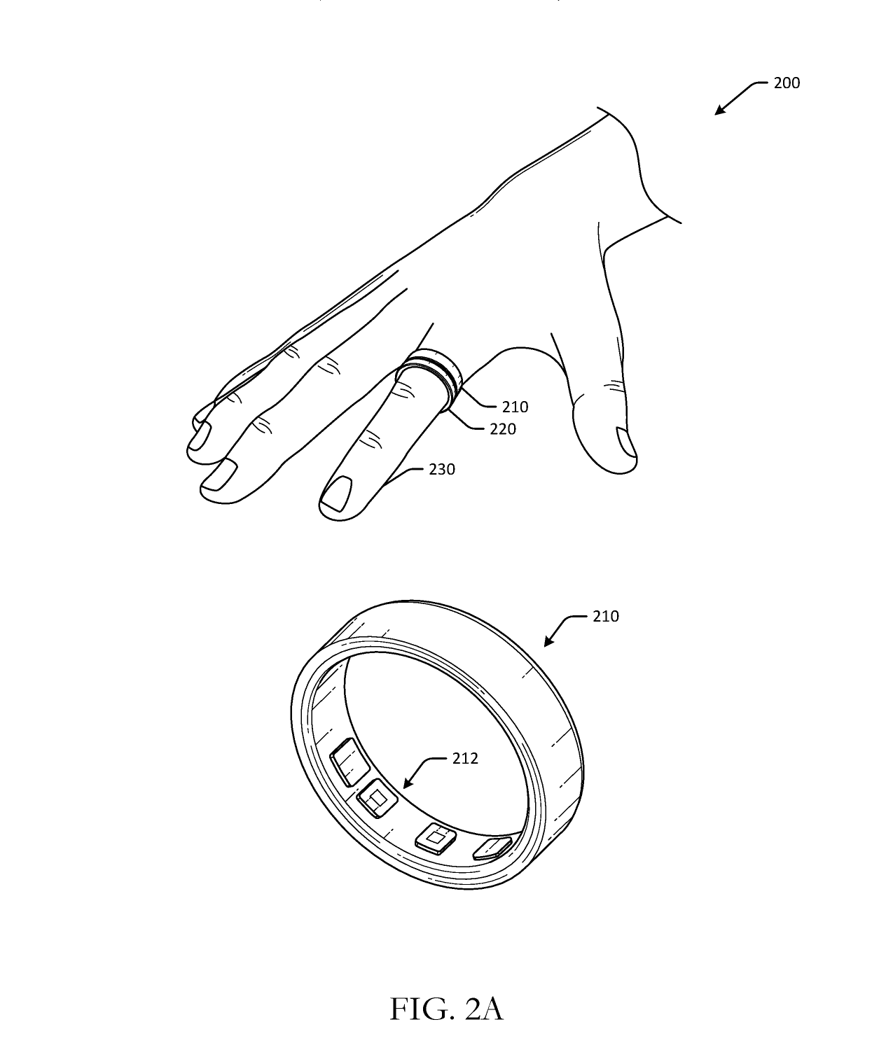

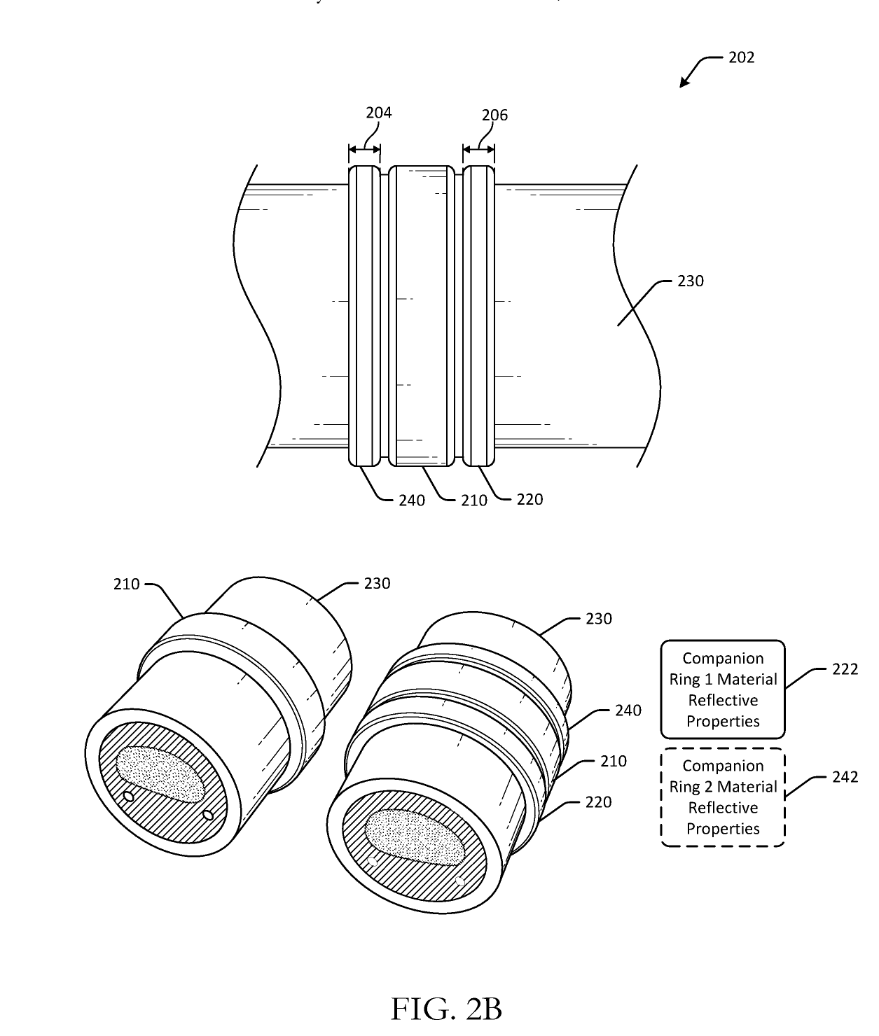

FIGS. 2A-2B are schematic illustrations of example use cases for active and passive companion rings for wearable devices in various configurations in accordance with one or more embodiments of the disclosure. Other embodiments may include additional or fewer components. The illustration of FIGS. 2A-2B may not be to scale, and may not be illustrated to scale with respect to other figures. The wearable devices and/or companion rings illustrated in FIGS. 2A-2B may be the wearable devices and/or companion rings discussed with respect to FIG. 1.

In a use case 200 of FIG. 2A, a user may be wearing a wearable ring device 210 and a companion ring 220 on an index finger 230. The wearable ring device 210 may be configured to detect one or more physiological metrics of the user via one or more optoelectronic components. For example, as depicted in isolated perspective view in FIG. 2A, the wearable ring device 210 may include one or more optoelectronic components 212, such as light emitting diodes of various colors, photodetectors, and/or other components that can be used to detect physiological metrics. In one example, light emitted from a light emitting diode can be detected using a photodetector, with an amount of light detected being indicative of a particular physiological metric. In particular, light emitting diodes that emit red light and green light may be included. Other embodiments may include additional, fewer, and/or different colors. The wearable ring device 210 may be a finger-worn wearable device, and have a housing having an interior diameter between 12 mm and 24 mm and an exterior diameter between 18 mm and 30 mm for at least a portion of the wearable ring device 210.

The companion ring 220 may be a passive companion ring and positioned adjacent to the wearable ring device 210. Light emitted from the light emitting diodes of the wearable ring device 210 may at least partially be reflected by the companion ring 220 and therefore impact the corresponding measurement, as the reflected light may not fully be a function of the finger tissue of the user 230 and/or allowed to escape through a side interface between the wearable ring device 210 and the finger 230. In other embodiments, the companion ring 220 may be an active companion ring and may include additional optoelectronic components, such as additional light emitting diodes of different colors, or additional battery capacity. For instance, in embodiments where the companion ring 220 includes additional battery capacity, the user may wear the companion ring 220 during exercise (e.g., jogging, etc.), so as to provide additional battery to the wearable ring device 210 and avoid depleting the battery of the wearable ring device 210.

In FIG. 2B, a top view 202 and partial cross-sectional view of the finger 230 of the user is depicted. In the top view 202, the user may wear more than one companion ring on the finger 230. The companion ring 220 may be a first companion ring and the user may wear a second companion ring 240 on an opposite side of the wearable ring device 210. Other configurations are possible and can be styled by the user.

The first companion ring 220 may have a first width 206 and the second companion ring 240 may have a second width 204. The respective widths of the companion rings may impact the effect the companion ring has on the amount of interference caused to measurements due to reflected light. For example, in many instances, the wider the companion ring, the greater the interference. In some embodiments, an optimized width for the companion ring(s) may be 4 mm to 5mm for instances where a width of the wearable ring device 210 is between 3 mm to 4 mm, and can vary depending on the width of the wearable ring device 210. Companion rings having widths greater than 5 mm may provide limited additional benefit to sensor performance.

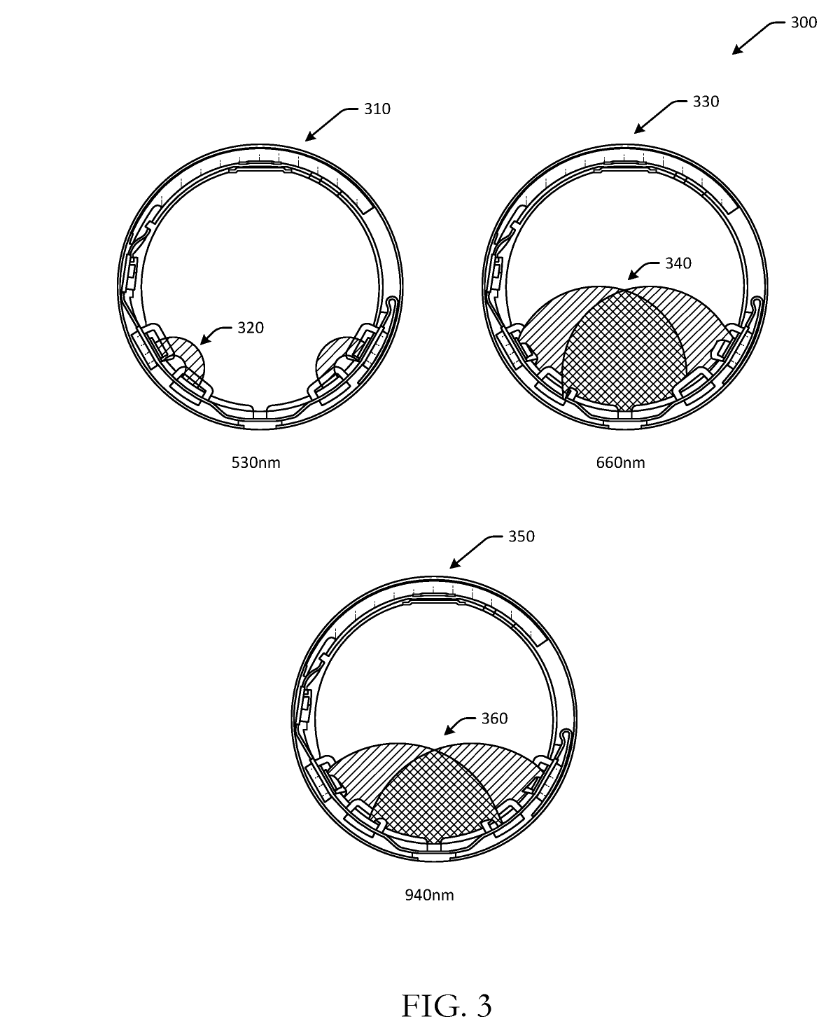

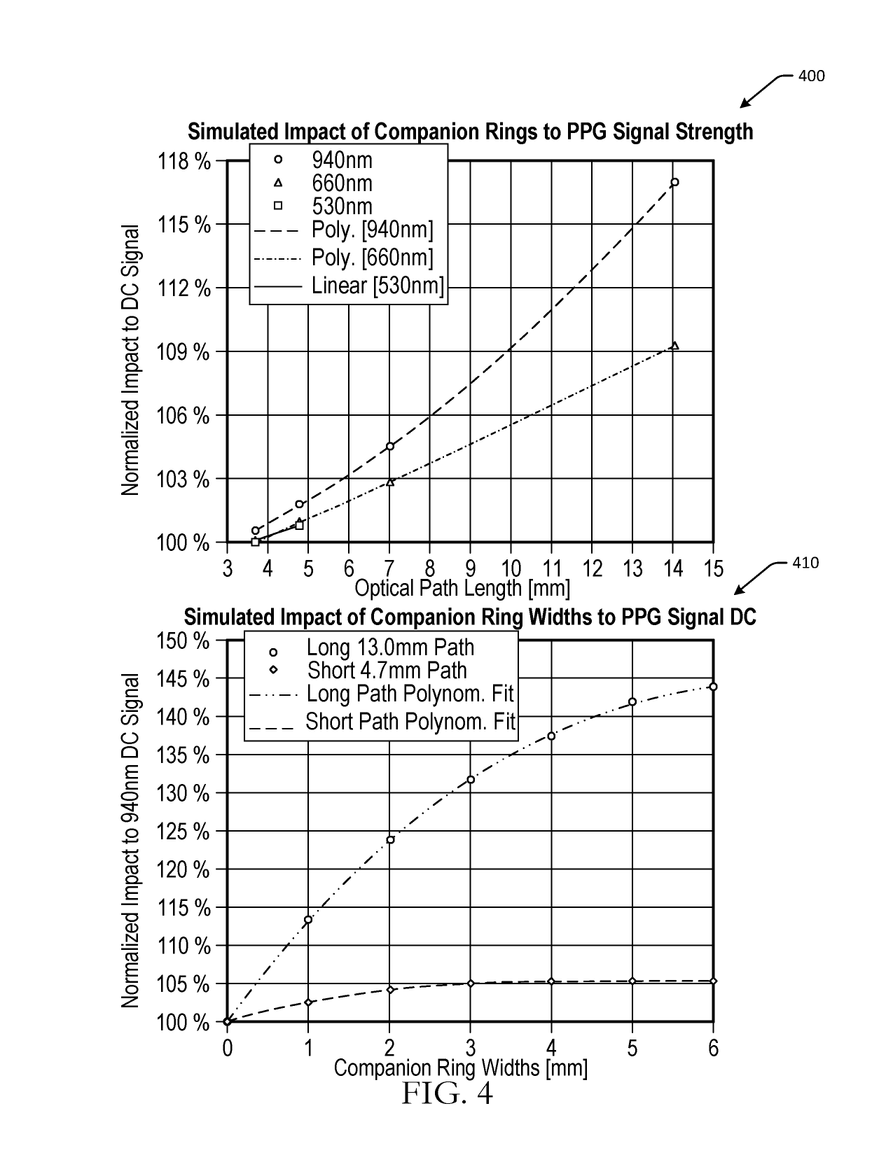

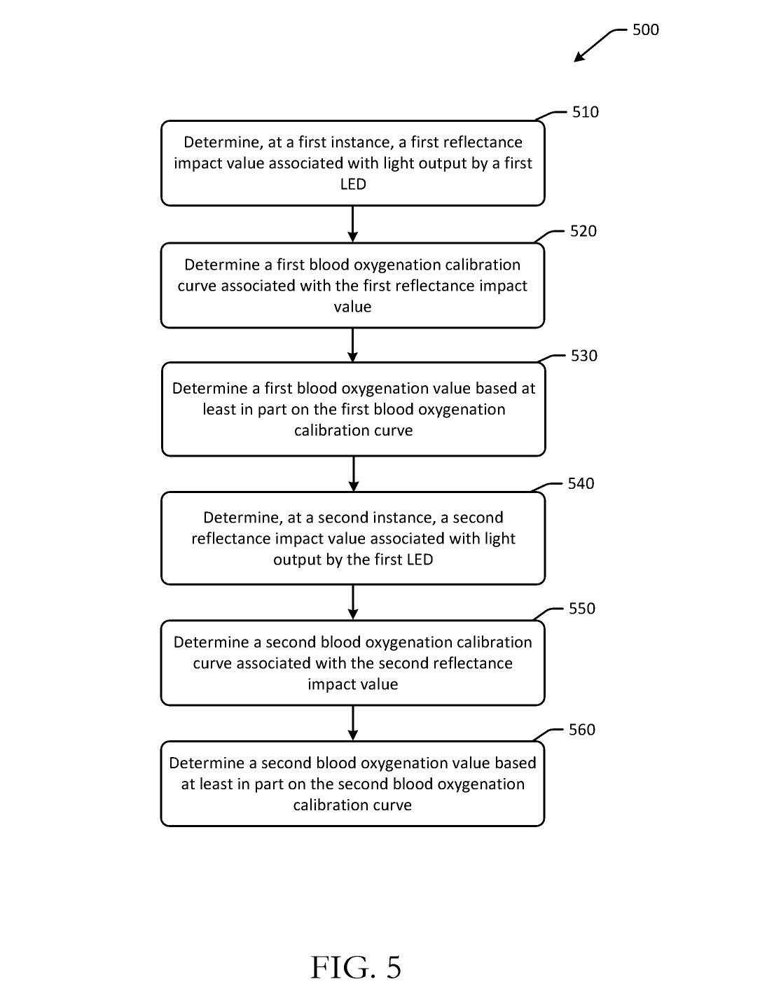

In addition, reflective properties of the material the respective companion ring is formed of can impact the amount of interference caused. The first companion ring 220 may have first material reflective properties 222 and the second companion ring 240 may have second material reflective properties 242. As discussed with respect to FIGS. 3-4, certain dimensions and/or materials may limit the interference caused by wearing companion rings adjacent to the wearable ring device 210, and in some instances, may enhance sensor performance of the wearable ring device 210.

For example, certain embodiments include companion rings, such as the first companion ring 220, that has a first housing or outer surface with a first surface reflectance value between about 5% and about 100%. Surface reflectance is the amount of light that reflects off a material's surface and can be represented as a ratio of reflected light relative to the light that impinges the surface. Reflectance from a companion ring can enhance optical signals and/or corresponding sensor measurements of the wearable ring device 210. The greater the surface reflectance value, the greater the potential signal enhancement. Accordingly, some embodiments include companion rings that have a surface reflectance value of 100%. Surface reflectance values may be optimized based on a light wavelength of light emitted by the light emitting diodes of the wearable ring device 210 and the corresponding sensing function. For example, a heart rate measurement performed with a single light wavelength benefits from as high reflectance as possible. In contrast, for blood oxygen saturation measurements, if light of two different wavelengths (e.g., red color and infrared, etc.) are used and compared to each other, the companion ring surface reflectance should be roughly equal for both wavelengths to avoid measurement error. For blood oxygen saturation, companion ring surface reflectance for companion rings should be optimized to be equal for both (or all) wavelengths, and absolute reflectance value can be a secondary consideration. Even for companion rings having surface reflectance values that are relatively low, such as about 5% (e.g., dark brown, black, etc.), interference may be limited but may still provide some enhancement. Certain embodiments may include companion rings having surface reflectance values of at least 50% and/or between 5% and 100%, or more specifically, between 50% and 100%.

Accordingly, the wearable ring device 210 may be a second ring removably coupled to the first ring (e.g., positioned adjacent to the first companion ring 220, etc.). The wearable ring device 210 may have a second housing with an outer portion having a first color and an inner portion having the first color in some instances. For example, the wearable ring device 210 may have a same color inner housing and outer housing, rather than two different colors. In some embodiments, the inner portion and the outer portion may be formed of the same material. The wearable ring device 210 may include a first light emitting diode configured to output green light, a second light emitting diode configured to output red light, and a photodiode or photodetector configured to detect light output from at least one of the first light emitting diode or the second light emitting diode. At least one of the wearable ring device 210 or the companion ring 220 can include a curved battery disposed in an upper half of the first housing or the second housing, respectively. The companion ring 220 may have an outer surface, which may be the first housing, that is formed of metal, and the first ring may have an exterior width between 4 mm and 5 mm. For instances where the user wears more than one companion ring, embodiments may include the second companion ring 240 with a third housing or outer surface having a second surface reflectance value between about 5% and about 100%. The second companion ring 240 may be disposed adjacent to a first side of the wearable ring device 210, and the first companion ring 220 may be disposed adjacent to a second side of the wearable ring device 210.

FIG. 3 is a schematic illustration of example wearable device sensor operation in accordance with one or more embodiments of the disclosure. Other embodiments may include additional or fewer components. The illustration of FIG. 3 is not to scale, and may not be illustrated to scale with respect to other figures. The wearable device illustrated in FIG. 3 may be the wearable devices discussed with respect to FIGS. 1-2B.

In FIG. 3, a wearable ring device 300 is depicted representing operation of various optoelectronic components. The wearable ring device 300 may have one or more optoelectronic light source components, such as light emitting diodes, vertical cavity surface emitting lasers, etc., and an optoelectronic component configured to detect light output from at least one of the one or more optoelectronic light source components, such as a photodiode or photodetector. Different optoelectronic components may be used for different biometric measurements. For example, for photoplethysmography measurements, a light source component may be used in conjunction with a light detection component to determine a measurement.

At a first instance 310, the wearable ring device 300 may include one or more light emitting diodes that emit green light 320 having a wavelength of 530 nanometers. Due to the relatively short wavelength of the green light, companion rings may have limited impact on corresponding measurements.

At a second instance 330, the wearable ring device 300 may include one or more light emitting diodes that emit red light 340 having a wavelength of 660 nanometers. In contrast to the green light, due to the relatively longer wavelength and higher tissue penetration depth of the red light, companion rings may have a greater impact on corresponding measurements. In one example, companion rings having a surface reflectance of about 50% or greater impact corresponding measurements by about 10%-20%, which can cause signal distortion if unaccounted for. Because blood oxygen saturation measurements rely on red and infrared light, such measurements may be particularly impacted.

At a third instance 350, the wearable ring device 300 may include one or more light emitting diodes that infrared light 360 having a wavelength of 940 nanometers. In contrast to the green light, and similar to red light, due to the relatively longer wavelength of the infrared light and higher tissue penetration depth, companion rings may have a greater impact on corresponding measurements. In one example, companion rings having a surface reflectance of about 50% or greater impact corresponding measurements by about 30%, which can cause signal distortion if unaccounted for. Because blood oxygen saturation measurements also rely on infrared light, such measurements may be particularly impacted.

FIG. 4 depicts graphs representing example data related to the simulated impact of companion rings to sensor data in accordance with one or more embodiments of the disclosure. The example graphs of FIG. 4 may relate to the wearable devices and/or companion rings discussed with respect to FIGS. 1-3.

In FIG. 4, a first graph 400 depicts the simulated impact to photoplethysmography signal strength by two passive companion rings positioned adjacent to both sides of a wearable ring device, with a normalized percentage impact to the direct current signal on a vertical axis and the optical path length in millimeters on the horizontal axis. The companion rings have a width of 3 mm and a surface reflectance value of 95%. The first graph 400 displays data for various light wavelengths, including 530 nm, 660 nm, and 940 nm.

For wearable ring devices with multiple optical path lengths, such as the four optical path lengths (e.g., approx. 3.75 mm, approx. 4.75 mm, approx. 7 mm, and approx. 14 mm, etc.) depicted in the first graph 400, the relationship between the optical path length and corresponding impact to signal is visible. The greater the optical path length, the greater the corresponding impact. Moreover, the impact increases in a non-linear manner as the optical path length increases, which indicates that longer optical path lengths are more sensitive to companion ring reflectance. In particular, for green light, the longer optical path between the two options has a greater direct current and greater corresponding impact, whereas the shorter optical path for green light, as well as red light, are not impacted or impacted by a reduced amount. As a result, the companion rings have a material impact to red light and infrared light signals due to the corresponding optical path lengths. The depicted maximum impact of 17% direct current increase is for infrared light. Such data can be specific to the design and dimensions of the wearable ring device itself, as well as to factors of the companion ring(s).

A second graph 410 depicts the simulated impact to photoplethysmography signal direct current by two companion rings positioned adjacent to both sides of a wearable ring device, with a normalized percentage impact to the 940 nm direct current signal on a vertical axis and the companion ring width in millimeters on the horizontal axis. The companion ring widths range from 0 mm (no companion ring) to 6 mm and a consistent surface reflectance value of 95%. The second graph 410 displays data for various optical path lengths, including 4.7 mm and 13.0 mm.

As evidenced by the second graph, different companion ring widths have different impacts. Companion ring width has a strong correlation with signal strength increase for 940 nm infrared light. For example, the infrared signal direct current at the long optical path increases by 13% with companion rings that are 1 mm wide, whereas 5 mm wide companion rings cause an increase of 42%. This is because as the companion ring width increases, the effective area of reflection increases as well. However, an upper threshold for impact can be noted as widths greater than about 6 mm do not appear to create additional impact. The upper threshold appears to be dependent on optical path length. For example, for short optical paths, the second graph 410 shows that when companion ring width is increased over 3mm, the impact to signal direct current has reached the upper threshold. Such data can be specific to the design and dimensions of the wearable ring device itself, as well as to factors of the companion ring(s). Accordingly, high surface reflectance values of companion ring materials can enhance signal strength, allowing for reduced power consumption and increased battery life for wearable ring devices. This can be particularly achievable for red and infrared light wavelengths with longer optical paths that can be used for measurements such as heart rate, blood oxygen saturation, etc.

Certain colors, materials, and/or coatings of companion rings may be preferable, such as rose gold color or material, depending on the type of light wavelength used and/or measurement being obtained. In contrast, a black coating on a wearable ring device may result in increased power consumption for a wearable ring device.

FIG. 5 is a schematic illustration of an example process flow 500 for automated calibration curve selection based on a detected companion ring in accordance with one or more example embodiments of the disclosure. While example embodiments of the disclosure may be described in the context of ring devices, it should be appreciated that the disclosure is more broadly applicable to any type of wearable device. Some or all of the blocks of the process flows in this disclosure may be performed in a distributed manner across any number of devices. The operations of the process flow 500 may be optional and may be performed in a different order.

In one example embodiment, the process flow 500 may be executed to automatically detect impact of a companion ring to sensor measurements by a wearable ring device, and to dynamically select a calibration curve to offset the impact. In this manner, regardless of what type of companion ring(s) a user wears or its corresponding surface reflectance value, or whether the user changes companion rings from time to time, measurement integrity can be maintained and any impact on sensor measurement accuracy can be mitigated. The process flow 500 may be executed by one or more components at a wearable ring device and/or connected device. The wearable ring device may include one or more optoelectronic light source components (e.g., multiple light emitting diodes, a single light emitting component with multiple light emitting chips in different colors, etc.), one or more optoelectronic light detection components (e.g., photodiode, etc.), and a controller.

At block 510 of the process flow 500, computer-executable instructions stored on a memory of a device, such as a wearable ring device, may be executed to determine, at a first instance, a first reflectance impact value associated with light output by a first light emitting diode or other optoelectronic light source component. For example, a control module executed at a controller associated with a wearable ring device may use one or more sensors to determine, at a first instance, a first reflectance impact value associated with light output by the first light emitting diode.

The wearable ring device may be a finger-worn wearable ring device configured to be worn adjacent to a companion ring having a first housing that comprises a first surface reflectance value between about 5% and about 100%. In one embodiment, the wearable ring device has a first light emitting diode configured to output green light and a second light emitting diode configured to output red light. Other embodiments may have additional, fewer, and/or different light emitting diodes or optoelectronic light source components. The wearable ring device may include a photodiode configured to detect light output from at least one of the first light emitting diode or the second light emitting diode. The wearable ring device may have an interior diameter between 12 mm and 24 mm and an exterior diameter between 18 mm and 30 mm.

The wearable ring device may determine the first reflectance impact value associated with light output by one of the optoelectronic light source components by executing a calibration process, which may include emitting light of different wavelengths and detecting reflected light. Such measurements may be compared to baseline values captured over time that are specific to a user. For embodiments that include multiple light emitting chips in a single light emitting diode component, calibration accuracy may be improved. Accordingly, the wearable ring device may determine reflectance impact value at a first instance or a first point in time for one or more light wavelengths. This process may be executed periodically or on demand to account for instances where a user removes a companion ring, adds a companion ring, changes a companion ring, and so forth, so as to maintain accurate sensor performance.

At block 520 of the process flow 500, computer-executable instructions stored on a memory of a device, such as a wearable ring device, may be executed to determine a first blood oxygen calibration curve associated with the first reflectance impact value. For example, a control module executed at a controller or computer system associated with a wearable ring device may determine a first blood oxygen calibration curve associated with the first reflectance impact value. In some embodiments, multiple calibration curves may be stored at the wearable device or at a device in communication with the wearable ring device. The calibration curves may be specific to certain light wavelengths and/or certain types of measurements, such as blood oxygen saturation. More than one set of calibration curves may be used for different light reflectance values and/or measurements. The individual calibration curves may be specific to different reflectance impact values (or ranges of reflectance impact values). Accordingly, the wearable ring device may determine or select a first blood oxygen calibration curve associated with the determined first reflectance impact value in the example of FIG. 5.

At block 530 of the process flow 500, computer-executable instructions stored on a memory of a device, such as a wearable ring device, may be executed to determine a first blood oxygen value based at least in part on the first blood oxygen calibration curve. For example, a control module executed at a controller or computer system associated with a wearable ring device may determine a first blood oxygen value based at least in part on the first blood oxygen calibration curve. Using the measured sensor data, the wearable ring device may offset the measurement by the corresponding calibration curve to determine an accurate value that accounts for impact of reflectance caused by companion ring(s). If no companion ring is used, the default sensor measurement may be used.

At block 540 of the process flow 500, computer-executable instructions stored on a memory of a device, such as a wearable ring device, may be executed to determine, at a second instance, a second reflectance impact value associated with light output by the first light emitting diode or other optoelectronic light source component. For example, a control module executed at a controller or computer system associated with a wearable ring device may determine, at a second instance, a second reflectance impact value associated with light output by the first light emitting diode. As users remove or replace companion rings, the reflectance impact value may change, and this process allows for updated selection of an accurate calibration curve. At block 550 of the process flow 500, computer-executable instructions stored on a memory of a device, such as a wearable ring device, may be executed to determine a second blood oxygen calibration curve associated with the second reflectance impact value. For example, a control module executed at a controller or computer system associated with a wearable ring device may determine a second blood oxygen calibration curve associated with the second reflectance impact value. As a result, sensor measurement integrity can be maintained regardless of the companion ring in use.

At block 560 of the process flow 500, computer-executable instructions stored on a memory of a device, such as a wearable ring device, may be executed to determine a second blood oxygen value based at least in part on the second blood oxygen calibration curve. For example, a control module executed at a controller or computer system associated with a wearable ring device may determine a second blood oxygen value based at least in part on the second blood oxygen calibration curve. By dynamically selecting calibration curves based on detected reflectance values, impact on sensor accuracy due to companion ring reflectance can be mitigated.

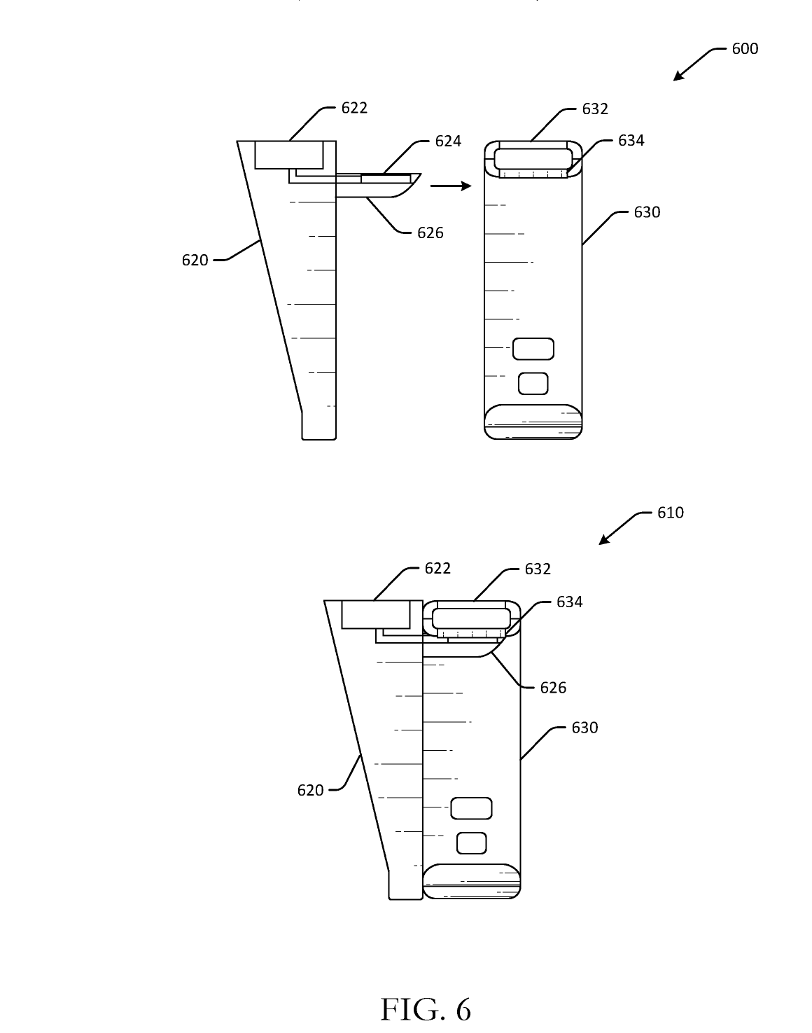

FIG. 6 depicts schematic illustrations of a wearable device and an active companion ring in both a coupled and an uncoupled configuration in accordance with one or more embodiments of the disclosure. Other embodiments may include additional or fewer components. The illustrations of FIG. 6 are not to scale, and may not be illustrated to scale with respect to other figures. The wearable devices and/or companion rings illustrated in FIG. 6 may be wearable devices and/or companion rings discussed with respect to FIGS. 1-5. In FIG. 6, a set of rings 600 may include a wearable ring device 630 and an example of an active companion ring 620. The active companion ring 620 may include one or more sensors, light sources, batteries, or other electronic components. In one example, the active companion ring 620 may be removably coupled to the wearable ring device 630 using one or more coupling components 626 having an electronic interface 624 that engages a port 634 formed in a housing 632 of the wearable ring device 630. The coupling components 626 may allow for electronic transmission of data and/or electric coupling for power transfer. Accordingly, in one embodiment, a first ring may have a first curved battery disposed in an upper half of a first housing, and a second ring may have a second curved battery disposed in an upper half of a second housing.

Such companion rings may allow for additional functionality, such as use of additional sensors, providing additional battery capacity for high battery discharge applications (e.g., measurements during exercise, etc.), allowing for reduced wearable ring device widths by reducing the amount of electronic components on a single wearable ring device, and so forth.

One or more operations of the methods, process flows, or use cases of FIGS. 1-6 may have been described above as being performed by a user device, or more specifically, by one or more program module(s), applications, or the like executing on a device. It should be appreciated, however, that any of the operations of the methods, process flows, or use cases of FIGS. 1-6 may be performed, at least in part, in a distributed manner by one or more other devices, or more specifically, by one or more program module(s), applications, or the like executing on such devices. In addition, it should be appreciated that processing performed in response to the execution of computer-executable instructions provided as part of an application, program module, or the like may be interchangeably described herein as being performed by the application or the program module itself or by a device on which the application, program module, or the like is executing. While the operations of the methods, process flows, or use cases of FIGS. 1-6 may be described in the context of the illustrative devices, it should be appreciated that such operations may be implemented in connection with numerous other device configurations.

The operations described and depicted in the illustrative methods, process flows, and use cases of FIGS. 1-6 may be carried out or performed in any suitable order, such as the depicted orders, as desired in various example embodiments of the disclosure. Additionally, in certain example embodiments, at least a portion of the operations may be carried out in parallel. Furthermore, in certain example embodiments, less, more, or different operations than those depicted in FIGS. 1-6 may be performed.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by the execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

### ILLUSTRATIVE COMPUTER ARCHITECTURE

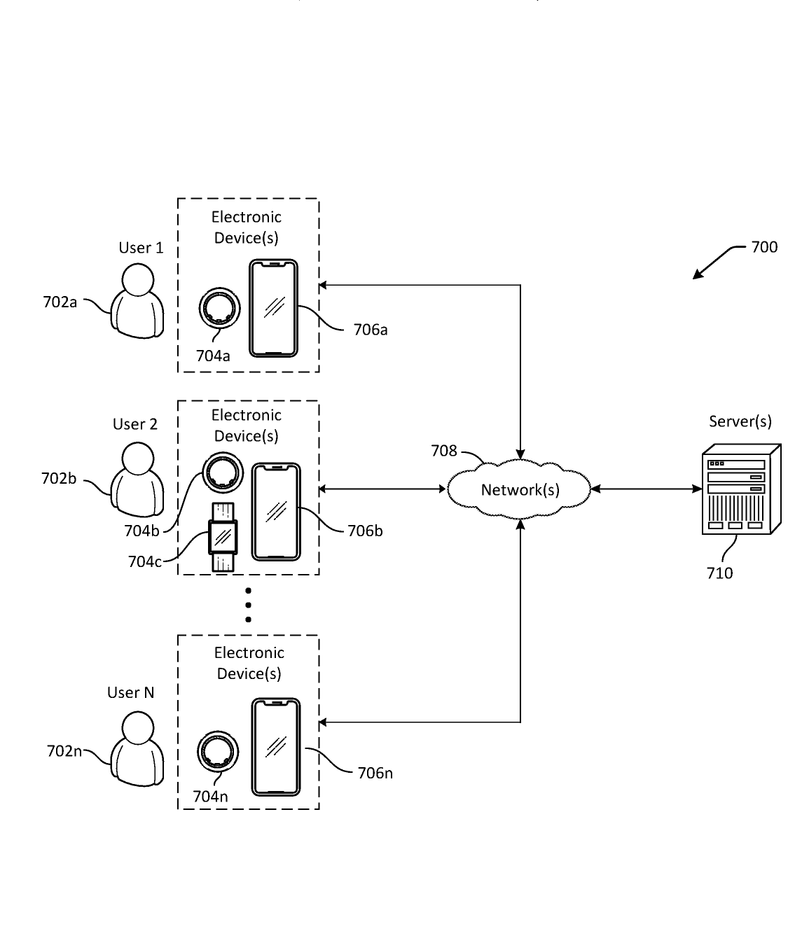

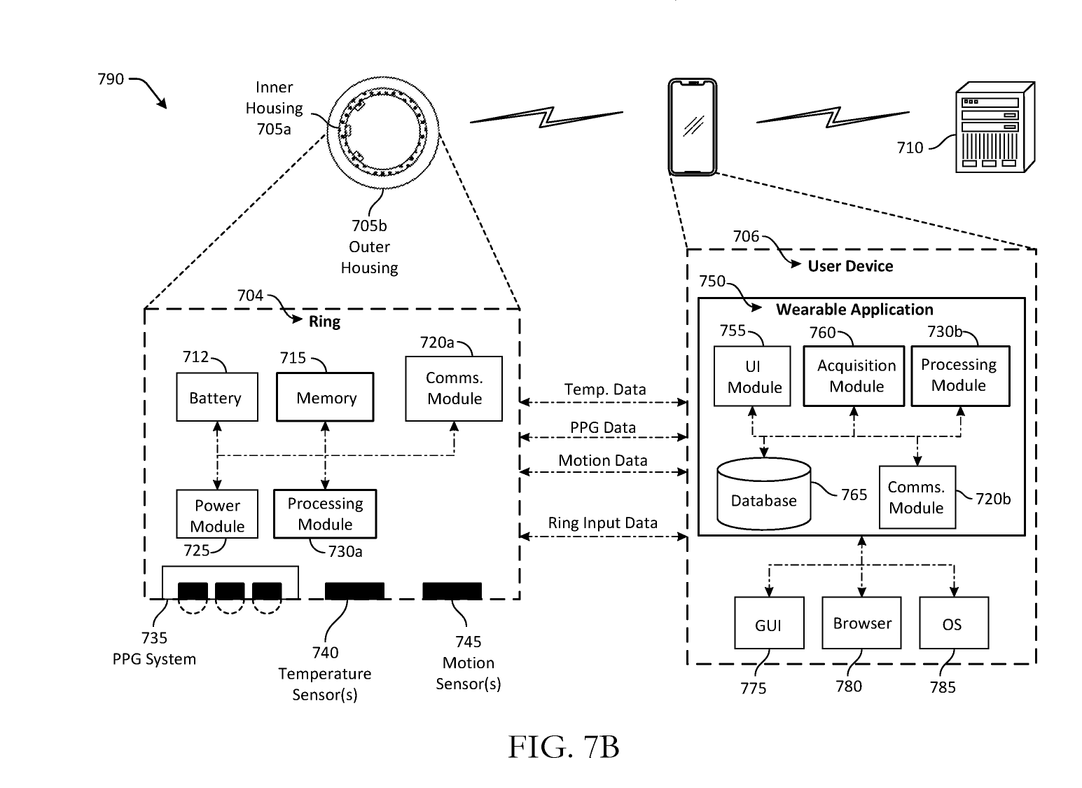

FIGS. 7A-7B schematically illustrate an example of a system 700 that supports techniques for active and passive companion ring detection for wearable devices in accordance with aspects of the present disclosure. The system 700 includes a plurality of electronic devices (e.g., wearable devices 704, user devices 706) that may be worn and/or operated by one or more users 702. The system 700 further includes a network 708 and one or more servers 710.

The electronic devices may include any electronic devices known in the art, including wearable devices 704 (e.g., ring wearable devices, watch wearable devices, etc.), user devices 706 (e.g., smartphones, laptops, tablets), and the like. The electronic devices associated with the respective users 702 may include one or more of the following functionalities: 1) measuring physiological data, 2) storing the measured data, 3) processing the data, 4) providing outputs (e.g., via GUIs and/or wirelessly coupled displays, etc.) to a user 702 based on the processed data, and 5) communicating data with one another and/or other computing devices. Different electronic devices may perform one or more of the functionalities.

Example wearable devices 704 may include wearable computing devices, such as a ring computing device (hereinafter "ring") configured to be worn on a user's 702 finger, a wrist computing device (e.g., a smart watch, fitness band, or bracelet) configured to be worn on a user's 702 wrist, and/or a head mounted computing device (e.g., glasses/goggles). Wearable devices 704 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 704 may also be attached to, or included in, articles of clothing. For example, wearable devices 704 may be included in pockets and/or pouches on clothing. As another example, wearable device 704 may be clipped and/or pinned to clothing, or may otherwise be maintained within the vicinity of the user 702. Example articles of clothing may include, but are not limited to, hats, shirts, gloves, pants, socks, outerwear (e.g., jackets), and undergarments. In some implementations, wearable devices 704 may be included with other types of devices such as training/sporting devices that are used during physical activity. For example, wearable devices 704 may be attached to, or included in, a bicycle, skis, a tennis racket, a golf club, and/or training weights.

Much of the present disclosure may be described in the context of a ring wearable device 704. Accordingly, the terms "ring 704," "wearable device 704," and like terms, may be used interchangeably, unless noted otherwise herein. However, the use of the term "ring 704" is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 706 may include handheld mobile computing devices, such as smartphones and tablet computing devices. User devices 706 may also include personal computers, such as laptop and desktop computing devices. Other example user devices 706 may include server computing devices that may communicate with other electronic devices (e.g., via wireless communication, via the Internet, etc.). In some implementations, computing devices may include medical devices, such as external wearable computing devices (e.g., Holter monitors). Medical devices may also include implantable medical devices, such as pacemakers and cardioverter defibrillators. Other example user devices 706 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 704, user devices 706) may measure physiological parameters of respective users 702, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some or all of the calculations or determinations described herein. Some electronic devices may not measure physiological parameters, but may perform some or all of the calculations or determinations described herein. For example, a ring (e.g., wearable device 704), mobile device application, or a server computing device may process received physiological data that was measured by other devices.

In some implementations, a user 702 may operate, or may be associated with, multiple electronic devices, some of which may measure physiological parameters and some of which may process the measured physiological parameters. In some implementations, a user 702 may have a ring (e.g., wearable device 704) that measures physiological parameters. The user 702 may also have, or be associated with, a user device 706 (e.g., mobile device, smartphone), where the wearable device 704 and the user device 706 are communicatively coupled to one another. In some cases, the user device 706 may receive data from the wearable device 704 and perform some or all of the calculations or determinations described herein. In some implementations, the user device 706 may also measure physiological parameters described herein, such as motion and/or activity parameters.

For example, as illustrated in FIG. 7A, a first user 702-a (User 1) may operate, or may be associated with, a wearable device 704-a (e.g., ring 704-a) and a user device 706-a that may operate as described herein. In this example, the user device 706-a associated with user 702-a may process and/or store physiological parameters measured by the ring 704-a. Comparatively, a second user 702-b (User 2) may be associated with a ring 704-b, a watch wearable device 704-c (e.g., watch 704-c), and a user device 706-b, where the user device 706-b associated with user 702-b may process and/or store physiological parameters measured by the ring 704-b and/or the watch 704-c. Moreover, an nth user 702-n (User N) may be associated with an arrangement of electronic devices described herein (e.g., ring 704-n, user device 706-n). In some aspects, wearable devices 704 (e.g., rings 704, watches 704) and other electronic devices may be communicatively coupled to the user devices 706 of the respective users 702 via Bluetooth, Wi- Fi, and other wireless protocols. Moreover, in some cases, the wearable device 704 and the user device 706 may be included within (or make up) the same device. For example, in some cases, the wearable device 704 may be configured to execute an application associated with the wearable device 704, and may be configured to display data via a GUI.

In some implementations, the rings 704 (e.g., wearable devices 704) of the system 700 may be configured to collect physiological data from the respective users 702 based on arterial blood flow within the user's finger. In particular, a ring 704 may utilize one or more light- emitting components, such as LEDs (e.g., red LEDs, green LEDs) that emit light on the palm- side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 700 may be configured to collect physiological data from the respective users 702 based on blood flow diffused into a microvascular bed of skin with capillaries and arterioles. For example, the system 700 may collect photoplethysmogram (PPG) data based on a measured amount of blood diffused into the microvascular system of capillaries and arterioles. In some implementations, the ring 704 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 704 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 704 has been found to exhibit superior performance as compared to wearable devices worn on the wrist, as the ring 704 may have greater access to arteries (as compared to capillaries), thereby resulting in stronger signals and more valuable physiological data.

The electronic devices of the system 700 (e.g., user devices 706, wearable devices 704) may be communicatively coupled to one or more servers 710 via wired or wireless communication protocols. For example, as shown in FIG. 7A, the electronic devices (e.g., user devices 706) may be communicatively coupled to one or more servers 710 via a network 708. The network 708 may implement transfer control protocol and internet protocol (TCP/IP), such as the Internet, or may implement other network 708 protocols. Network connections between the network 708 and the respective electronic devices may facilitate transport of data via email, web, text messages, mail, or any other appropriate form of interaction within a computer network 708. For example, in some implementations, the ring 704-a associated with the first user 702-a may be communicatively coupled to the user device 706-a, where the user device706-a is communicatively coupled to the servers 710 via the network 708. In additional or alternative cases, wearable devices 704 (e.g., rings 704, watches 704) may be directly communicatively coupled to the network 708.

The system 700 may offer an on-demand database service between the user devices 706 and the one or more servers 710. In some cases, the servers 710 may receive data from the user devices 706 via the network 708, and may store and analyze the data. Similarly, the servers 710 may provide data to the user devices 706 via the network 708. In some cases, the servers 710 may be located at one or more data centers. The servers 710 may be used for data storage, management, and processing. In some implementations, the servers 710 may provide a web- based interface to the user device 706 via web browsers.

In some aspects, the system 700 may detect periods of time that a user 702 is asleep, and classify periods of time that the user 702 is asleep into one or more sleep stages (e.g., sleep stage classification). For example, as shown in FIG. 7A, User 702-a may be associated with a wearable device 704-a (e.g., ring 704-a) and a user device 706-a. In this example, the ring 704-a may collect physiological data associated with the user 702-a, including temperature, heart rate, HRV, respiratory rate, and the like. In some aspects, data collected by the ring 704-a may be input to a machine learning classifier, where the machine learning classifier is configured to determine periods of time that the user 702-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 702-a via a GUI of the user device 706-a. Sleep stage classification may be used to provide feedback to a user 702-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 700 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 702-a via the wearable device 704-a. In this example, the circadian rhythm adjustment model may be configured to "weight," or adjust, physiological data collected throughout a user's natural, approximately 24-hour circadian rhythm. In some implementations, the system may initially start with a "baseline" circadian rhythm adjustment model, and may modify the baseline model using physiological data collected from each user 702 to generate tailored, individualized circadian rhythm adjustment models that are specific to each respective user 702.

In some aspects, the system 700 may utilize other biological rhythms to further improve physiological data collection, analysis, and processing by phase of these other rhythms. For example, if a weekly rhythm is detected within an individual's baseline data, then the model may be configured to adjust "weights" of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day "weeks," 12 day rhythms could be used); 5) multi-day ovarian rhythms in women and spermatogenesis rhythms in men; 6) lunar rhythms (relevant for individuals living with low or no artificial lights); and 7) seasonal rhythms. The biological rh^{y}thms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 700 may support techniques for active and passive companion ring detection for wearable devices. For example, certain calibration curves may be selected based on detection of companion ring material properties, so as to minimize impact to sensor data and related measurements.

It should be appreciated by a person skilled in the art that one or more aspects of the disclosure may be implemented in a system 700 to additionally or alternatively solve other problems than those described above. Furthermore, aspects of the disclosure may provide technical improvements to "conventional" systems or processes as described herein. However, the description and appended drawings only include example technical improvements resulting from implementing aspects of the disclosure, and accordingly do not represent all of the technical improvements provided within the scope of the claims.

FIG. 7B illustrates an example of a system 790 that supports techniques for active and passive companion ring detection for wearable devices in accordance with aspects of the present disclosure. The system 790 may implement, or be implemented by, system 700. In particular, system 790 illustrates an example of a ring 704 (e.g., wearable device 704), a user device 706, and a server 710, as described with reference to FIG. 7A.

In some aspects, the ring 704 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 790 further includes a user device 706 (e.g., a smartphone) in communication with the ring 704. For example, the ring 704 may be in wireless and/or wired communication with the user device 706. In some implementations, the ring 704 may send measured and processed data (e.g., temperature data, PPG data, motion/accelerometer data, ring input data, and the like) to the user device 706. The user device 706 may also send data to the ring 704, such as ring 704 firmware/configuration updates. The user device 706 may process data. In some implementations, the user device 706 may transmit data to the server 710 for processing and/or storage.

The ring 704 may include a housing 705 that may include an inner housing 705-a and an outer housing 705-b. In some aspects, the housing 705 of the ring 704 may store or otherwise include various components of the ring including, but not limited to, device electronics, a power source (e.g., battery 712, and/or capacitor), one or more substrates (e.g., printable circuit boards) that interconnect the device electronics and/or power source, and the like. The device electronics may include device modules (e.g., hardware/software), such as: a processing module 730-a, a memory 715, a communication module 720-a, a power module 725, and the like. The device electronics may also include one or more sensors. Example sensors may include one or more temperature sensors 740, a PPG sensor assembly (e.g., PPG system 735), and one or more motion sensors 745.

The sensors may include associated modules configured to communicate with the respective components/modules of the ring 704, and generate signals associated with the respective sensors. In some aspects, some or all of the components/modules of the ring 704 may be communicatively coupled to one another via wired or wireless connections. Moreover, the ring 704 may include additional and/or alternative sensors or other components that are configured to collect physiological data from the user, including light sensors (e.g., LEDs), oximeters, and the like.

The ring 704 shown and described with reference to FIG. 7B is provided solely for illustrative purposes. As such, the ring 704 may include additional and/or different components as those illustrated in FIG. 7B. Other rings 704 that provide functionality described herein may be fabricated. For example, rings 704 with fewer components (e.g., sensors) may be fabricated. In a specific example, a ring 704 with a single temperature sensor 740 (or other sensor), a power source, and device electronics configured to read the single temperature sensor 740 (or other sensor) may be fabricated. In another specific example, a temperature sensor 740 (or other sensor) may be coupled to a user's finger (e.g., using adhesives, wraps, clamps, spring loaded clamps, etc.). In this case, the sensor may be wired to another computing device, such as a wrist worn computing device that reads the temperature sensor 740 (or other sensor). In other examples, a ring 704 that includes additional sensors and processing functionality may be fabricated.

The housing 705 may include the outer housing 705-b component (e.g., a shell) and the inner housing 705-a component (e.g., a molding). The housing 705 may include additional components (e.g., additional layers). For example, in some implementations, the ring 704 may include one or more insulating layers that electrically insulate the device electronics and other conductive materials (e.g., electrical traces) from the outer housing 705-b (e.g., a metal outer housing 705-b). The housing 705 may provide structural support for the device electronics, battery 712, substrate(s), and other components. For example, the housing 705 may protect the device electronics, battery 712, and substrate(s) from mechanical forces, such as pressure and impacts. The housing 705 may also protect the device electronics, battery 712, and substrate(s) from water and/or other chemicals, as well as provide a barrier against liquid ingress.

The outer housing 705-b may be fabricated from one or more materials. In some implementations, the outer housing 705-b may include a metal, such as titanium, that may provide strength and abrasion resistance at a relatively light weight. The outer housing 705-b may also be fabricated from other materials, such polymers. In some implementations, the outer housing 705-b may be protective as well as decorative.

The inner housing 705-a may be configured to interface with the user's finger. The inner housing 705-a may be formed from a polymer (e.g., a medical grade polymer) or other material. In some implementations, the inner housing 705-a may be transparent. For example, the inner housing 705-a may be transparent or translucent with respect to light emitted by the PPG light emitting diodes (LEDs). In some implementations the inner housing 705-a component may be mold onto the outer housing 705-b. For example, the inner housing 705-a may include a polymer that is molded (e.g., injection molded) to fit into an outer housing 705-b metallic shell.

The ring 704 may include one or more substrates (not illustrated). The device electronics and battery 712 may be included on the one or more substrates. For example, the device electronics and battery 712 may be mounted on one or more substrates. Example substrates may include one or more PCBs, such as flexible PCB (e.g., polyimide). In some implementations, the electronics/battery 712 may include surface mounted devices (e.g., surface- mount technology (SMT) devices) on a flexible PCB. In some implementations, the one or more substrates (e.g., one or more flexible PCBs) may include electrical traces that provide electrical communication between device electronics. The electrical traces may also connect the battery 712 to the device electronics.

The various components/modules of the ring 704 represent functionality (e.g., circuits and other components) that may be included in the ring 704. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The device electronics, battery 712, and substrates may be arranged in the ring 704 in a variety of ways. In some implementations, one substrate that includes device electronics may be mounted along the bottom of the ring 704 (e.g., the bottom half), such that the sensors (e.g., PPG system 735, temperature sensors 740, motion sensors 745, and other sensors) interface with the underside of the user's finger. In these implementations, the battery 712 may be included along the top portion of the ring 704 (e.g., on another substrate).

The memory 715 (memory module) of the ring 704 may include any volatile, non- volatile, magnetic, or electrical media, such as a random access memory (RAM), read-only memory (ROM), non-volatile RAM (NVRAM), electrically-erasable programmable ROM (EEPROM), flash memory, or any other memory device. The memory 715 may store any of the data described herein. For example, the memory 715 may be configured to store data (e.g., motion data, temperature data, PPG data) collected by the respective sensors and PPG system 735. Furthermore, memory 715 may include instructions that, when executed by one or more processing circuits, cause the modules to perform various functions attributed to the modules herein. The device electronics of the ring 704 described herein are only example device electronics. As such, the types of electronic components used to implement the device electronics may vary based on design considerations.

The functions attributed to the modules of the ring 704 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 730-a of the ring 704 may include one or more processors (e.g., processing units), microcontrollers, digital signal processors, systems on a chip (SOCs), and/or other processing devices. The processing module 730-a communicates with the modules included in the ring 704. For example, the processing module 730-a may transmit/receive data to/from the modules and other components of the ring 704, such as the sensors. As described herein, the modules may be implemented by various circuit components. Accordingly, the modules may also be referred to as circuits (e.g., a communication circuit and power circuit).

The processing module 730-a may communicate with the memory 715. The memory 715 may include computer-readable instructions that, when executed by the processing module 730-a, cause the processing module 730-a to perform the various functions attributed to the processing module 730-a herein. In some implementations, the processing module 730-a (e.g., a microcontroller) may include additional features associated with other modules, such as communication functionality provided by the communication module 720-a (e.g., an integrated Bluetooth Low Energy transceiver) and/or additional onboard memory 715.

The communication module 720-a may include circuits that provide wireless and/or wire communication with the user device 706 (e.g., communication module 720-b of the user device 706). In some implementations, the communications modules 720-a, 720-b may include wireless communication circuits, such as Bluetooth circuits and/or Wi-Fi circuits. In some implementations, the communication modules 720-a, 720-b can include wired communication circuits, such as Universal Serial Bus (USB) communication circuits. Using the communication module 720-a, the ring 704 and the user device 706 may be configured to communicate with each other. The processing module 730-a of the ring may be configured to transmit/receive data to/from the user device 706 via the communication module 720-a. Example data may include, but is not limited to, motion data, temperature data, pulse waveforms, heart rate data, HRV data, PPG data, and status updates (e.g., charging status, battery charge level, and/or ring 704 configuration settings). The processing module 730-a of the ring may also be configured to receive data from the user device 706, such as updates (e.g., software/firmware updates).

The ring 704 may include a battery 712 (e.g., a rechargeable battery 712). An example battery 712 may include a Lithium-Ion or Lithium-Polymer type battery 712, although a variety of battery 712 options are possible. The battery 712 may be wirelessly charged. In some implementations, the ring 704 may include a power source other than the battery 712, such as a capacitor. The power source (e.g., battery 712 or capacitor) may have a curved geometry that matches the curve of the ring 704. In some aspects, a charger or other power source may include additional sensors that may be used to collect data in addition to, or that supplements, data collected by the ring 704 itself. Moreover, a charger or other power source for the ring 704 may function as a user device 706, in which case the charger or other power source for the ring 704 may be configured to receive data from the ring 704, store and/or process data received from the ring 704, and communicate data between the ring 704 and the servers 710.

In some aspects, the ring 704 includes a power module 725 that may control charging of the battery 712. For example, the power module 725 may interface with an external wireless charger that charges the battery 712 when interfaced with the ring 704. The charger may include a datum structure that mates with a ring 704 datum structure to create a specified orientation with the ring 704 during charging. The power module 725 may also regulate voltage(s) of the device electronics, regulate power output to the device electronics, and monitor the state of charge of the battery 712. In some implementations, the battery 712 may include a protection circuit module (PCM) that protects the battery 712 from high current discharge, over voltage during charging, and under voltage during discharge.

The one or more temperature sensors 740 may be electrically coupled to the processing module 730-a. The temperature sensor 740 may be configured to generate a temperature signal (e.g., temperature data) that indicates a temperature read or sensed by the temperature sensor 740. The processing module 730-a may determine a temperature of the user in the location of the temperature sensor 740. For example, in the ring 704, temperature data generated by the temperature sensor 740 may indicate a temperature of a user at the user's finger (e.g., skin temperature). In some implementations, the temperature sensor 740 may contact the user's skin. In other implementations, a portion of the housing 705 (e.g., the inner housing 705-a) may form a barrier (e.g., a thin, thermally conductive barrier) between the temperature sensor 740 and the user's skin. In some implementations, portions of the ring 704 configured to contact the user's finger may have thermally conductive portions and thermally insulative portions. The thermally conductive portions may conduct heat from the user's finger to the temperature sensors 740. The thermally insulative portions may insulate portions of the ring 704 (e.g., the temperature sensor 740) from ambient temperature.

In some implementations, the temperature sensor 740 may generate a digital signal (e.g., temperature data) that the processing module 730-a may use to determine the temperature. As another example, in cases where the temperature sensor 740 includes a passive sensor, the processing module 730-a (or a temperature sensor 740 module) may measure a current and/or voltage generated by the temperature sensor 740 and determine the temperature based on the measured current and/or voltage. Example temperature sensors 740 may include a thermistor, such as a negative temperature coefficient (NTC) thermistor, or other types of sensors including resistors, transistors, diodes, and/or other electrical/electronic components

The processing module 730-a may sample the user's temperature over time. For example, the processing module 730-a may sample the user's temperature according to a sampling rate. An example sampling rate may include one sample per second, although the processing module 730-a may be configured to sample the temperature signal at other sampling rates that are higher or lower than one sample per second. In some implementations, the processing module 730-a may sample the user's temperature continuously throughout the day and night. Sampling at a sufficient rate (e.g., one sample per second) throughout the day may provide sufficient temperature data for analysis described herein.

The processing module 730-a may store the sampled temperature data in memory 715. In some implementations, the processing module 730-a may process the sampled temperature data. For example, the processing module 730-a may determine average temperature values over a period of time. In one example, the processing module 730-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 715 may store the average temperature values over time. In some implementations, the memory 715 may store average temperatures (e.g., one per minute) instead of sampled temperatures in order to conserve memory 715.

The sampling rate, which may be stored in memory 715, may be configurable. In some implementations, the sampling rate may be the same throughout the day and night. In other implementations, the sampling rate may be changed throughout the day/night. In some implementations, the ring 704 may filter/reject temperature readings, such as large spikes in temperature that are not indicative of physiological changes (e.g., a temperature spike from a hot shower). In some implementations, the ring 704 may filter/reject temperature readings that may not be reliable due to other factors, such as excessive motion during exercise (e.g., as indicated by a motion sensor 745).

The ring 704 (e.g., communication module) may transmit the sampled and/or average temperature data to the user device 706 for storage and/or further processing. The user device 706 may transfer the sampled and/or average temperature data to the server 710 for storage and/or further processing.

Although the ring 704 is illustrated as including a single temperature sensor 740, the ring 704 may include multiple temperature sensors 740 in one or more locations, such as arranged along the inner housing 705-a near the user's finger. In some implementations, the temperature sensors 740 may be stand-alone temperature sensors 740. Additionally, or alternatively, one or more temperature sensors 740 may be included with other components (e.g., packaged with other components), such as with the accelerometer and/or processor.

The processing module 730-a may acquire and process data from multiple temperature sensors 740 in a similar manner described with respect to a single temperature sensor 740. For example, the processing module 730 may individually sample, average, and store temperature data from each of the multiple temperature sensors 740. In other examples, the processing module 730-a may sample the sensors at different rates and average/store different values for the different sensors. In some implementations, the processing module 730-a may be configured to determine a single temperature based on the average of two or more temperatures determined by two or more temperature sensors 740 in different locations on the finger

The temperature sensors 740 on the ring 704 may acquire distal temperatures at the user's finger (e.g., any finger). For example, one or more temperature sensors 740 on the ring 704 may acquire a user's temperature from the underside of a finger or at a different location on the finger. In some implementations, the ring 704 may continuously acquire distal temperature (e.g., at a sampling rate). Although distal temperature measured by a ring 704 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a "shell" temperature) may differ from the user's core temperature. As such, the ring 704 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 704 may include a PPG system 735. The PPG system 735 may include one or more optical transmitters that transmit light. The PPG system 735 may also include one or more optical receivers that receive light transmitted by the one or more optical transmitters. An optical receiver may generate a signal (hereinafter "PPG" signal) that indicates an amount of light received by the optical receiver. The optical transmitters may illuminate a region of the user's finger. The PPG signal generated by the PPG system 735 may indicate the perfusion of blood in the illuminated region. For example, the PPG signal may indicate blood volume changes in the illuminated region caused by a user's pulse pressure. The processing module 730-a may sample the PPG signal and determine a user's pulse waveform based on the PPG signal. The processing module 730-a may determine a variety of physiological parameters based on the user's pulse waveform, such as a user's respiratory rate, heart rate, HRV, oxygen saturation, and other circulatory parameters.

In some implementations, the PPG system 735 may be configured as a reflective PPG system 735 where the optical receiver(s) receive transmitted light that is reflected through the region of the user's finger. In some implementations, the PPG system 735 may be configured as a transmissive PPG system 735 where the optical transmitter(s) and optical receiver(s) are arranged opposite to one another, such that light is transmitted directly through a portion of the user's finger to the optical receiver(s).

The number and ratio of transmitters and receivers included in the PPG system 735 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 735.

The PPG system 735 illustrated in FIG. 7B may include a reflective PPG system 735 in some implementations. In these implementations, the PPG system 735 may include a centrally located optical receiver (e.g., at the bottom of the ring 704) and two optical transmitters located on each side of the optical receiver. In this implementation, the PPG system 735 (e.g., optical receiver) may generate the PPG signal based on light received from one or both of the optical transmitters. In other implementations, other placements, combinations, and/or configurations of one or more optical transmitters and/or optical receivers are contemplated.

The processing module 730-a may control one or both of the optical transmitters to transmit light while sampling the PPG signal generated by the optical receiver. In some implementations, the processing module 730-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 735 may result in a pulse waveform that may be referred to as a "PPG." The pulse waveform may indicate blood pressure over time for multiple cardiac cycles. The pulse waveform may include peaks that indicate cardiac cycles. Additionally, the pulse waveform may include respiratory induced variations that may be used to determine respiration rate. The processing module 730-a may store the pulse waveform in memory 715 in some implementations. The processing module 730-a may process the pulse waveform as it is generated and/or from memory 715 to determine user physiological parameters described herein.

The processing module 730-a may determine the user's heart rate based on the pulse waveform. For example, the processing module 730-a may determine heart rate (e.g., in beats per minute) based on the time between peaks in the pulse waveform. The time between peaks may be referred to as an interbeat interval (IBI). The processing module 730-a may store the determined heart rate values and IBI values in memory 715. The processing module 730-a may determine HRV over time. For example, the processing module 730-a may determine HRV based on the variation in the IBIs. The processing module 730-a may store the HRV values over time in the memory 715. Moreover, the processing module 730-a may determine the user's respiratory rate over time. For example, the processing module 730-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 730-a may store user respiratory rate values over time in the memory 715.

The ring 704 may include one or more motion sensors 745, such as one or more accelerometers (e.g., 6-D accelerometers) and/or one or more gyroscopes (gyros). The motion sensors 745 may generate motion signals that indicate motion of the sensors. For example, the ring 704 may include one or more accelerometers that generate acceleration signals that indicate acceleration of the accelerometers. As another example, the ring 704 may include one or more gyro sensors that generate gyro signals that indicate angular motion (e.g., angular velocity) and/or changes in orientation. The motion sensors 745 may be included in one or more sensor packages. An example accelerometer/gyro sensor is a BOSCH® BM1160 inertial micro electro- mechanical system (MEMS) sensor that may measure angular rates and accelerations in three perpendicular axes.

The processing module 730-a may sample the motion signals at a sampling rate (e.g., 50Hz) and determine the motion of the ring 704 based on the sampled motion signals. For example, the processing module 730-a may sample acceleration signals to determine acceleration of the ring 704. As another example, the processing module 730-a may sample a gyro signal to determine angular motion. In some implementations, the processing module 730-a may store motion data in memory 715. Motion data may include sampled motion data as well as motion data that is calculated based on the sampled motion signals (e.g., acceleration and angular values).

The ring 704 may store a variety of data described herein. For example, the ring 704 may store temperature data, such as raw sampled temperature data and calculated temperature data (e.g., average temperatures). As another example, the ring 704 may store PPG signal data, such as pulse waveforms and data calculated based on the pulse waveforms (e.g., heart rate values, IBI values, HRV values, and respiratory rate values). The ring 704 may also store motion data, such as sampled motion data that indicates linear and angular motion.

The ring 704, or other computing device, may calculate and store additional values based on the sampled/calculated physiological data. For example, the processing module 730 may calculate and store various metrics, such as sleep metrics (e.g., a Sleep Score), activity metrics, and readiness metrics. In some implementations, additional values/metrics may be referred to as "derived values." The ring 704, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 704 is oriented on the user's finger and if the ring 704 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 730-a may compress the data stored in memory 715. For example, the processing module 730-a may delete sampled data after making calculations based on the sampled data. As another example, the processing module 730-a may average data over longer periods of time in order to reduce the number of stored values. In a specific example, if average temperatures for a user over one minute are stored in memory 715, the processing module 730-a may calculate average temperatures over a five minute time period for storage, and then subsequently erase the one minute average temperature data. The processing module 730-a may compress data based on a variety of factors, such as the total amount of used/available memory 715 and/or an elapsed time since the ring 704 last transmitted the data to the user device 706.

Although a user's physiological parameters may be measured by sensors included on a ring 704, other devices may measure a user's physiological parameters. For example, although a user's temperature may be measured by a temperature sensor 740 included in a ring 704, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 704 can make physiological measurements in a resting/sleep state in order to acquire cleaner physiological signals. In one example, the ring 704 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 704 may be configured to collect, store, and/or process data, and may transfer any of the data described herein to the user device 706 for storage and/or processing. In some aspects, the user device 706 includes a wearable application 750, an operating system (OS), a web browser application (e.g., web browser 780), one or more additional applications, and a GUI 775. The user device 706 may further include other modules and components, including sensors, audio devices, haptic feedback devices, and the like. The wearable application 750 may include an example of an application (e.g., "app") that may be installed on the user device 706. The wearable application 750 may be configured to acquire data from the ring 704, store the acquired data, and process the acquired data as described herein. For example, the wearable application 750 may include a user interface (UI) module 755, an acquisition module 760, a processing module 730-b, a communication module 720-b, and a storage module (e.g., database 765) configured to store application data.

In some cases, the wearable device 704 and the user device 706 may be included within (or make up) the same device. For example, in some cases, the wearable device 704 may be configured to execute the wearable application 750, and may be configured to display data via the GUI 775.

The various data processing operations described herein may be performed by the ring 704, the user device 706, the servers 710, or any combination thereof. For example, in some cases, data collected by the ring 704 may be pre-processed and transmitted to the user device 706. In this example, the user device 706 may perform some data processing operations on the received data, may transmit the data to the servers 710 for data processing, or both. For instance, in some cases, the user device 706 may perform processing operations that require relatively low processing power and/or operations that require a relatively low latency, whereas the user device 706 may transmit the data to the servers 710 for processing operations that require relatively high processing power and/or operations that may allow relatively higher latency.

In some aspects, the ring 704, user device 706, and server 710 of the system 790 may be configured to evaluate sleep patterns for a user. In particular, the respective components of the system 790 may be used to collect data from a user via the ring 704, and generate one or more scores (e.g., Sleep Score, Readiness Score) for the user based on the collected data. For example, as noted previously herein, the ring 704 of the system 790 may be worn by a user to collect data from the user, including temperature, heart rate, HRV, and the like. Data collected by the ring 704 may be used to determine when the user is asleep in order to evaluate the user's sleep for a given "sleep day." In some aspects, scores may be calculated for the user for each respective sleep day, such that a first sleep day is associated with a first set of scores, and a second sleep day is associated with a second set of scores. Scores may be calculated for each respective sleep day based on data collected by the ring 704 during the respective sleep day. Scores may include, but are not limited to, Sleep Scores, Readiness Scores, and the like.

In some cases, "sleep days" may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a "cut-off time," where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 790 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more "contributors," "factors," or "contributing factors." For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The "total sleep" contributor may refer to the sum of all sleep periods of the sleep day. The "efficiency" contributor may reflect the percentage of time spent asleep compared to time spent awake while in bed, and may be calculated using the efficiency average of long sleep periods (e.g., primary sleep period) of the sleep day, weighted by a duration of each sleep period. The "restfulness" contributor may indicate how restful the user's sleep is, and may be calculated using the average of all sleep periods of the sleep day, weighted by a duration of each period. The restfulness contributor may be based on a "wake up count" (e.g., sum of all the wake-ups (when user wakes up) detected during different sleep periods), excessive movement, and a "got up count" (e.g., sum of all the got-ups (when user gets out of bed) detected during the different sleep periods).

The "REM sleep" contributor may refer to a sum total of REM sleep durations across all sleep periods of the sleep day including REM sleep. Similarly, the "deep sleep" contributor may refer to a sum total of deep sleep durations across all sleep periods of the sleep day including deep sleep. The "latency" contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the "timing" contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The "sleep" contributor may refer to the combined Sleep Score of all sleep periods within the sleep day. The "sleep balance" contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The "resting heart rate" contributor may indicate a lowest heart rate from the longest sleep period of the sleep day (e.g., primary sleep period) and/or the lowest heart rate from naps occurring after the primary sleep period.

Continuing with reference to the "contributors" (e.g., factors, contributing factors) of the Readiness Score, the "HRV balance" contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The "recovery index" contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The "body temperature" contributor may be calculated based on the longest sleep period (e.g., primary sleep period) or based on a nap happening after the longest sleep period if the user's highest temperature during the nap is at least 0.5°C higher (or another value) than the highest temperature during the longest period. In some aspects, the ring may measure a user's body temperature while the user is asleep, and the system 790 may display the user's average temperature relative to the user's baseline temperature. If a user's body temperature is outside of their normal range (e.g., clearly above or below 0.0 or another value), the body temperature contributor may be highlighted (e.g., go to a "Pay attention" state) or otherwise generate an alert for the user.

What is claimed is:

1. A set of finger-worn wearable ring devices comprising:
a first ring having a first housing, wherein the housing comprises a first surface reflectance value between about 5% and about 100%; and
a second ring removably coupled to the first ring, the second ring comprising:
a second housing comprising an outer portion having a first color and an inner portion having the first color;
a first light emitting diode (LED) configured to output green light;
a second LED configured to output red light; and
a photodiode configured to detect light output from at least one of the first LED, or the second LED.

2. The set of finger-worn wearable ring devices of claim 1, wherein at least one of the first ring or the second ring comprises a curved battery disposed in an upper half of the first housing or the second housing, respectively.

3. The set of finger-worn wearable ring devices of claim 1, wherein the first housing is formed of metal, and wherein the first ring comprises an exterior width between 4 mm and 5 mm.

4. The set of finger-worn wearable ring devices of claim 1, wherein the second ring further comprises:
a photodetector; and
a controller configured to:
determine, at a first instance, a first reflectance impact value associated with light output by the second LED;
determine a first blood oxygenation calibration curve associated with the first reflectance impact value;
determine a first blood oxygenation value based at least in part on the first blood oxygenation calibration curve;
determine, at a second instance, a second reflectance impact value associated with light output by the second LED;
determine a second blood oxygenation calibration curve associated with the second reflectance impact value; and
determine a second blood oxygenation value based at least in part on the second blood oxygenation calibration curve.

5. A set of wearable ring devices comprising:
a first ring having a first housing, wherein the housing comprises a first surface reflectance value between about 5% and about 100%; and
a second ring disposed adjacent to the first ring, the second ring comprising:
a second housing;
one or more optoelectronic light source components; and
an optoelectronic component configured to detect light output from at least one of the one or more optoelectronic light source components.

6. The set of wearable ring devices of claim 5, wherein the second housing comprises an outer portion having a first color and an inner portion having the first color.

7. The set of wearable ring devices of claim 5, wherein the one or more optoelectronic components comprise a first light emitting diode (LED) and a second LED, and wherein the second ring further comprises:
a controller configured to:
determine, at a first instance, a first reflectance impact value associated with light output by the second LED;
determine a first blood oxygenation calibration curve associated with the first reflectance impact value;
determine a first blood oxygenation value based at least in part on the first blood oxygenation calibration curve;
determine, at a second instance, a second reflectance impact value associated with light output by the second LED;
determine a second blood oxygenation calibration curve associated with the second reflectance impact value; and
determine a second blood oxygenation value based at least in part on the second blood oxygenation calibration curve.

8. The set of wearable ring devices of claim 5, further comprising:
a third ring having a third housing, wherein the third housing comprises a second surface reflectance value between about 5% and about 100%;
wherein the third ring is disposed adjacent to a first side of the second ring, and the first ring is disposed adjacent to a second side of the second ring.

9. The set of wearable ring devices of claim 5, wherein the first housing is formed of metal.

10. The set of wearable ring devices of claim 5, wherein the first ring comprises an exterior width between 4 mm and 5 mm.

11. The set of wearable ring devices of claim 5, wherein the first ring is removably coupled to the second ring.

12. The set of wearable ring devices of claim 5, wherein the second ring is a finger-worn wearable device, and wherein the second housing has an interior diameter between 12 mm and 24 mm and an exterior diameter between 18 mm and 30 mm for at least a portion of the second ring.

13. The set of wearable ring devices of claim 5, wherein at least one of the first ring or the second ring comprises a curved battery disposed in an upper half of the first housing or the second housing, respectively.

14. The set of wearable ring devices of claim 5, wherein the first ring comprises a first curved battery disposed in an upper half of the first housing, and the second ring comprises a second curved battery disposed in an upper half of the second housing.

15. A finger-worn wearable ring device configured to be worn adjacent to a first ring having a first housing that comprises a first surface reflectance value between about 5% and about 100%, the finger-worn wearable ring device comprising:
a second housing comprising an outer portion having a first color and an inner portion having the first color;
a first light emitting diode (LED) configured to output green light;
a second LED configured to output red light; and
a photodiode configured to detect light output from at least one of the first LED or the second LED.

16. The finger-worn wearable ring device of claim 15, further comprising:
a photodetector; and
a controller configured to:
determine, at a first instance, a first reflectance impact value associated with light output by the second LED;
determine a first blood oxygenation calibration curve associated with the first reflectance impact value;
determine a first blood oxygenation value based at least in part on the first blood oxygenation calibration curve;
determine, at a second instance, a second reflectance impact value associated with light output by the second LED;
determine a second blood oxygenation calibration curve associated with the second reflectance impact value; and
determine a second blood oxygenation value based at least in part on the second blood oxygenation calibration curve.

17. The finger-worn wearable ring device of claim 15, wherein the second housing is formed of metal, and the first ring comprises an exterior width between 4 mm and 5 mm.

18. The finger-worn wearable ring device of claim 15, wherein the first ring is removably coupled to the finger-worn wearable ring device.

19. The finger-worn wearable ring device of claim 15, further comprising:
a curved battery disposed in an upper half of the second housing.

20. The finger-worn wearable ring device of claim 15, wherein the second housing has an interior diameter between 12 mm and 24 mm and an exterior diameter between 18 mm and 30 mm for at least a portion of the second ring.
